(19) United States
(12) Patent Application Publication
(10) Pub. No.: US 2021/0346885 A1
(43) Pub. Date: Nov. 11, 2021

(54) CELLULAR ANALYTIC SYSTEMS

(71) Applicant: Hewlett-Packard Development Company, L.P., Spring, TX (US)

(72) Inventors: Alexander Govyadinov, Corvallis, OR (US); Viktor Shkolnikov, Palo Alto, CA (US); Diane R. Hammerstad, Corvallis, OR (US)

(73) Assignee: Hewlett-Packard Development Company, L.P., Spring, TX (US)

(21) Appl. No.: 17/059,650

(22) PCT Filed: Feb. 1, 2019
(86) PCT No.: PCT/US2019/016321

§ 371 Date: Nov. 30, 2020

(51) Int. Cl. B01L 3/00; G01N 15/00

(52) U.S. Cl. CPC B01L 3/502715; B01L 3/50273; G01N 15/00; B01L 2400/0475; B01L 2300/0627; B01L 2300/087; G01N 2015/0069

(57) ABSTRACT

In one example in accordance with the present disclosure, a cellular analytic system is described. The cellular analytic system includes an analytic device. The analytic device includes a chamber to receive a cell to be analyzed. At least one lysing element agitates the cell and at least one sensor detects a change in the cell based on an agitation of the cell. The cellular analytic system also includes a controller to determine a rupture threshold of the cell based on parameters of the agitation when a cell membrane ruptures.

### BACKGROUND

Analytic chemistry is a field of chemistry that uses instruments to separate, identify, and quantify matter. Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell membrane and frees the inner components. The fluid resulting from the bursting of the cell is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a cellular analytic system for cell rupture threshold determination, according to an example of the principles described herein.

FIG. 2 is a flow chart of a method of cell rupture threshold determination, according to an example of the principles described herein.

FIG. 3 is a diagrammatic representation of a cellular analytic system for cell rupture threshold determination, according to an example of the principles described herein.

FIG. 4 is a diagrammatic representation of a cellular analytic system for cell rupture threshold determination, according to another example of the principles described herein.

FIG. 5 is a diagrammatic representation of a cellular analytic system for cell rupture threshold determination, according to another example of the principles described herein.

FIG. 6 is a diagrammatic representation of a cellular analytic system for cell rupture threshold determination, according to another example of the principles described herein.

FIG. 7 is a diagrammatic representation of a cellular analytic system for cell rupture threshold determination, according to another example of the principles described herein.

FIG. 8 is a diagrammatic representation of a cellular analytic system for cell rupture threshold determination, according to another example of the principles described herein.

FIG. 9 is a diagrammatic representation of a cellular analytic system for cell rupture threshold determination, according to another example of the principles described herein.

FIG. 10 is a diagrammatic representation of a cellular analytic system for cell rupture threshold determination, according to another example of the principles described herein.

FIG. 11 is a flow chart of a method of cell rupture threshold determination, according to another example of the principles described herein.

FIG. 12 is a diagrammatic representation of a cellular analytic system for cell rupture threshold determination, according to another example of the principles described herein.

FIG. 13 is a diagrammatic representation of a cellular analytic system for cell rupture threshold determination, according to another example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cellular analytics is a field of chemistry that uses instruments to separate, identify, and quantify matter. A wealth of information can be collected during cellular analytics, specifically relating to the mechanical properties of the cell membrane and even more specifically relating to the mechanical breakdown of the cell membrane. In some examples, the physical characteristics of a cell can be used to classify and/or differentiate a particular cell from other cells. In another example, changes to the physical characteristics of a cell can be used to determine a state of the cell. For example, parasitic invasion of a cell—such as occurs in cells affected by malaria—can alter the membrane of the cell. Gross changes to tissue, such as when a cell is cancerous, can also alter the physical properties of the cell membrane. However, while the rupture threshold of a cell may aide in classifying or otherwise analyzing the cell, current methods are deficient in determining a rupture threshold for a cell. That is current methods cannot characterize cells based on the rupture threshold of the cell. In other words, as described above a rupture threshold of a cell can provide a wealth of information used to classify a cell and current methods, by not being able to determine a rupture threshold, thereby fail to provide any of this valuable information.

For example, to test cell membrane characteristics, some methods may deform a cell, for example via a cantilever that is pressed against the cell. The amount of deformation is then physically observed and cell properties determined based on an amount of force used to deform the cell. However, such analysis is ineffective as the deformation is localized to the area where the cell was pressed, and information gleaned therefrom may not be representative of the characteristics of the entire cell membrane.

Moreover, while other forms of cell cytometry exist, such analyses do not determine cellular rupture thresholds and thereby do not present an accurate or effective way to glean information that may be based on a cell membrane's properties.

Accordingly, the present specification describes systems and methods that provide a new method of cell characterization which has not previously been available. That is, the new method can characterize a cell via a mechanical rupture threshold. As it is a new cell characterization method, it thereby provides information not previously available. That is, a cell membrane strength is a fundamental cell property and is a function of what proteins, lipids, and cholesterol is solvated into the membrane. These characteristics vary from cell type to cell type and thus the present method can differentiate and classify cells based on these properties.

Accordingly, the present specification describes systems and methods for analyzing a rupture threshold of a cell using a destructive method. By so doing, cellular characteristics and cellular differentiating information can be collected from the cell. For example, changes to a cell membrane over time can be detected by rupturing the cell membrane and determining whether it ruptures earlier or later than expected, that is whether the cell membrane ruptures under a lower stress than expected. In another example, differences in the stress under which different cell membranes rupture can be used to classify those different cells. In other words, information may be gleaned from a cellular sample by rupturing the cell membrane and then analyzing information related to the cell rupture, such as an intensity of the force that caused the rupture.

Accordingly, the present specification describes a method of flow cytometry which senses cell presence. A lysing element or multiple lysing elements and corresponding sensors then gradually increase a stress applied to a cell until the cell ruptures, which point is defined as the cell rupture threshold.

Specifically, the present specification describes a cellular analytic system. The cellular analytic system includes an analytic device that includes 1) a chamber to receive a cell to be analyzed, 2) at least one lysing element to agitate the cell, and 3) at least one sensor to detect a change in the cell based on an agitation of the cell. The cellular analytic system also includes a controller to determine a rupture threshold of the cell based on parameters of the agitation when a cell membrane ruptures.

The present specification also describes a method. According to the method, a cell to be analyzed is received at a cellular analytic system. The cell is exposed to repeated agitation cycles within increasing intensity. This is done until the cell ruptures. It is then determined, based on a number of agitation cycles and an intensity of each agitation cycle, a rupture threshold of the cell.

The present specification also describes an example of a cellular analytic system that includes an analytic device. The analytic device includes 1) at least one chamber to receive a cell to be analyzed, 2) at least one lysing element, and 3) at least one sensor per lysing element to determine when the cell ruptures. In this example, the analytic device repeatedly agitates the cell with increasing intensity until the cell ruptures. The cellular analytic system also includes a main pump to move the cell through the analytic device and a controller. The controller includes 1) a count determiner to determine a number of agitation cycles until cell rupture, 2) an intensity determiner to determine an intensity of each of the number of agitation cycles, 3) a threshold determiner to determine a rupture threshold of the cell based on the number of agitation cycles until cell rupture and the intensity of each of the number of agitation cycles, and 4) a component controller to alter operation of at least one component of the analytic device based on a cell rupture.

In summary, using such a cellular analytic system 1) allows analysis of the cell rupture threshold; 2) leads to cell classification/differentiation based on the cell rupture threshold; 3) automates precision separation and cytometry 4) can be integrated onto a lab-on-a-chip device; 5) is scalable and can be parallelized, and 6) is low cost and effective. However, the devices disclosed herein may address other matters and deficiencies in a number of technical areas.

As used in the present specification and in the appended claims, the term “cell membrane” refers to any enclosing structure of a cell, organelle, or other cellular particle.

Further, as used in the present specification and in the appended claims, the term “agitation cycle” refers to a period when a cell is exposed to the operations of a lysing element. For example, an agitation cycle may refer to each time a cell is looped past a single lysing element. That is, exposing the cell to repeated agitation cycles comprises looping the cell past a single lysing element. In another example, a cell passes through an agitation cycle each time it passes by a lysing element in a string of multiple lysing elements. That is, exposing the cell to repeated agitation cycles comprises moving the cell past multiple lysing elements.

Even further, as used in the present specification and in the appended claims, the term “rupture threshold” refers to the amount of stress that a cell can withstand before rupturing. In other words, the rupture threshold is the threshold at which the cell ruptures. The rupture threshold may be determined based on any number of factors including a number of agitation cycles a cell is exposed to and the intensity of the agitation cycles.

Yet further, as used in the present specification and in the appended claims, the term “parameters” refers to the operating conditions in a particular agitation cycle. Accordingly, different “parameters” may mean different lysing element types and/or different lysing strengths. For example, agitation parameters for an agitation cycle may include whether a lysing element is a thermal inkjet resistor, a piezo-electric device, or an ultrasonic transducer. Agitation parameters also refer to the operating conditions of the particular lysing element. For example, the parameters of an ultrasonic transducer may refer to the frequency, amplitude, and/or phase of ultrasonic waves. The parameters of the thermal inkjet resistor and piezo-electric device may refer to the size of the element and/or the voltage applied to the element.

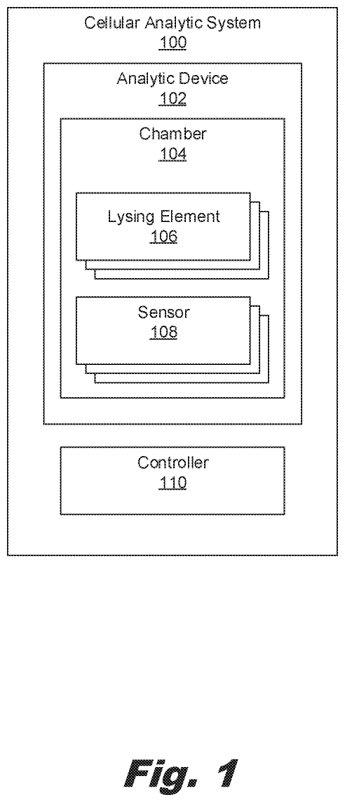

Turning now to the figures, FIG. 1 is a block diagram of a cellular analytic system (100) for cell rupture threshold determination, according to an example of the principles described herein. As described herein, the cellular analytic system (100) is a collection of components for analyzing a cell and for determining a rupture threshold of the cell. In some examples, the cellular analytic system (100) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small scale fluid analysis functionality.

In other words, the components, i.e., the analytic devices (102), chamber (104), lysing elements (106), and sensors (108) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

In general, the cellular analytic system includes an analytic device (102). The analytic device includes a chamber (104) to receive a cell to be analyzed and at least one lysing element (106) to agitate the cell. The analytic device (102) also includes at least one sensor (108) to detect a change in the cell based on an agitation of the cell. A controller (110) of the cellular analytic system (100) determines a rupture threshold of the cell based on parameters of the agitation when a cell membrane ruptures.

The cellular analytic system (100) includes an analytic device (102) which performs the cellular analysis. Accordingly, the analytic device (102) includes a variety of sub-components. Specifically, the analytic device (102) includes a chamber (104) where lysing and lysis detection occur. In some examples, the chamber (104) includes a single lysing element (106) and sensor (108). In other examples, the chamber (104) includes multiple lysing elements (106) and sensors (108).

In some examples the chamber (104) may be no more than 100 times a volume of a cell to be lysed. In other examples, the chamber (104) may have a cross-sectional size comparable with the cell size and in some cases smaller than the cell so as to deform the cell before or during the rupturing of the cell membrane. That is, the chamber (104) may be a microfluidic structure. As the chamber (104) is the location where lysis occurs, the chamber (104) receives a cell or other component to be lysed. In some examples, the chamber (104) may receive the cells single-file, or serially. Thus, lysing operations can be performed on a single cell and that cell's particular properties may be analyzed and processed.

The analytic device (102) also includes at least one lysing element (106) to agitate the cell. Lysis refers to the agitation of a cell with the objective of rupturing a cell membrane. The lysing element (106) may implement any number of agitation mechanisms, including shearing, ball milling, pestle grinding, and using rotating blades to grind the membranes. Other examples of agitation mechanisms include localized heating and shearing by constriction. In another example, repeated cycles of freezing and thawing can disrupt cells through ice crystal formation. Solution-based lysis is yet another example. In these examples, the osmotic pressure in the cellular particle could be increased or decreased to collapse the cell membrane or to cause the membrane to burst. As yet another example, the cells may be forced through a narrow space, thereby shearing the cell membranes.

In one example, the lysing element (106) is a thermal inkjet heating resistor disposed within a microfluidic channel. In this example, the thermal inkjet resistor heats up in response to an applied current. As the resistor heats up, a portion of the fluid in the chamber (104) vaporizes to generate a bubble. This bubble generates a pressure and shear spike which rupture the cell membrane.

In another example, the lysing element (106) may be a piezoelectric device. As a voltage is applied, the piezoelectric device changes shape which generates a pressure pulse in the chamber (104) that generates a pressure and shear spike which rupture the cell membrane.

In yet another example, the lysing element (106) may be a non-reversible electroporation electrode that forms nano-scale pores on the cell membrane. These pores grow and envelope the entire cell membrane leading to membrane lysis. While particular examples of lysing elements (106) have been described herein, a variety of lysing element (106) types may be implemented in accordance with the principles described herein.

In yet another example, the lysing element (106) is an ultrasonic transducer that generates high energy sonic waves. These high energy waves may travel through the wall of the chamber (104) to shear the cells disposed therein.

The different types of lysing elements (106) each may exhibit a different agitation mechanism. For example, the agitation mechanism of an ultrasonic transducer is the ultrasonic waves that are emitted and that shear the cells. The agitation mechanism of the thermal inkjet heating resistor is the vapor bubble that is generated and ruptures the cell membrane. The agitation mechanism of the piezo-electric device is the pressure wave that is generated during deformation of the piezo-electric device, which pressure wave shears the cell membrane.

As described above, the cellular analytic system (100) of the present specification gradually increases lysing intensity. Accordingly, in one example this may mean passing the cell by a single lysing element (106) multiple times. With each cycle, the lysing intensity may or may not change. Based on the number of cycles, a rupture threshold of the cell may be determined. For example, if a cell is intact following 10 agitation cycles, but breaks down following the eleventh agitation cycle, the rupture threshold may be determined accordingly.

In another example, increasing the lysing intensity may include passing the cell past multiple lysing elements (106). That is, a chamber (104) may include multiple lysing elements (106) placed along a flow path. Those lysing elements (106) downstream may have an increasingly intense lysing strength such that a rupture threshold for a cell may be determined based on where along the flow path the cell membrane ruptured.

To determine when a cell has ruptured, the analytic device (102) may include at least one sensor (108). That is, the at least one sensor (108) detects a change in the cell based on an agitation of the cell by the at least one lysing element (106).

The sensor (108) may take many forms. For example, the cell sensor (108) may be an optical scatter sensor that determines cell rupture based on a scatter of reflected energy waves. The cell sensor (108) may be an optical fluorescence sensor that detects cell rupture based on the detection of certain fluorescent markers. In other examples, the sensor (108) may be an optical bright field imaging system, an optical dark field imaging system, or a thermal property sensor.

In one particular example, the sensor (108) is an impedance sensor. Specifically, the sensor (108) may include at least one pair of electrodes spaced apart from one another by a gap. These electrodes detect a level of conductivity within the gap. That is, incoming cells to a chamber (104), and the solution in which they are contained, have a predetermined electrical conductivity. Any change to the contents of the chamber (104) will effectively change the electrical conductivity within the chamber (104). Specifically, as the cells are ruptured and the nucleic acid pours out, the conductivity would increase. To measure the conductivity, a resistance of solution between electrodes of the impedance sensor is measured and a conductivity determined therefrom. In some examples, a single pair of electrodes are used, with one electrode plate placed at either end of a chamber (104). In another example, multiple pair of electrodes are used. For example, one pair of electrode plates could be placed at the inlet and another pair of electrode plates placed at the outlet.

Thus in summary, the sensor (108) which may include one sensor (108) in the chamber (104) or which may include multiple sensors (108) in a multi-lysing element (106) chamber (104), can determine when a cell membrane has been ruptured. This information is passed to a controller (110) which determines a rupture threshold of the cell based on the parameters of the agitation when the cell membrane ruptures. That is, as described above a cell may be exposed to gradually increasing intensities of lysing operations. The characteristics of the different agitation cycles can be passed to the controller (110) which determines a rupture threshold. The controller (110) may also use this information to perform other operations. For example, the controller (110) may differentiate cells in a sample based on different rupture thresholds. In this example, the controller (110) may receive, for multiple cells, information regarding the results of lysing by different lysing element(s) (106) on those cells. Based on the results, the controller (110) may determine when each cell in a sample is ruptured. Different types of cells may rupture under different intensities. Accordingly, based on when a cell ruptures, the controller (110) may be able to determine the cell types of the various cells in a sample.

As another example, the controller (110) may be able to determine a state of a cellular sample. For example, it may be determined that healthy cells rupture at a particular lysing intensity. This may be determined by passing healthy cells through the cellular analytic system (100) and collecting rupturing information from the sensor(s) (108). Accordingly, a sample to be analyzed may subsequently be passed through the cellular analytic system (100) and rupturing information collected for these cells in the sample. If the rupturing information indicates that the sample cells rupture at a lower intensity than the healthy cells, the controller (110) may determine that the sample cells are diseased.

As yet another example, the controller (110) may be able to differentiate between live cells and dead cells based on the rupturing thresholds of different cells as determined by the analytic device (102). That is, live cells may be more robust against lysing and therefore have a higher rupturing threshold as compared to dead cells which may rupture at a lower intensity.

In addition to determining a rupture threshold, the controller (110) may also be used to determine when the cell membrane has ruptured based on detected levels of conductivity in the chamber (104). That is, the controller (110) may compare detected levels of conductivity within the chamber (104) with a threshold level of conductivity associated with a ruptured cell. Accordingly, once the detected level of conductivity within the chamber has reached the threshold value, the controller (110) may determine that a cell has been ruptured.

In a specific example, the chamber (104) may have a 20 by 20 micrometer cross section with an electrode separation of 360 micrometers. In this example, voltages may be applied in the range of 5 to 80 V incrementally to determine a cell rupture threshold.

In a specific use of the cellular analytic system (100) a cell may be introduced into the chamber (104) and the lysing element (106) activated to a first intensity. The sensor (108) may determine whether or not the cell ruptured. If not, the cell may be repositioned below the lysing element (106) and the energy increased or maintained. In either case, the sensor (108) again determines whether the cell ruptured or not. This continues until it is determined that the cell has ruptured. Throughout all of this, information such as agitation cycle count and the intensity of each agitation cycle is passed to the controller (110). Once ruptured, the fact that the cell ruptured, the number of agitation cycles, and the overall intensity of each cycle is used by the controller (110) to determine a characteristic of the cell, such as if it is diseased or not, or whether it is alive or dead, among others.

Thus, the present cellular analytic system (100) provides an incremental way of determining with accuracy and precision a rupture threshold for a cell. That is, it can be precisely determined what the rupturing threshold for a cell is. Such precision allows for precise and fine-tuned differentiation and classification of cells in a sample. Moreover, the cellular analytic system (100) as described herein allows for effective analysis of cell membrane properties which provide a wealth of cellular information. For example, the cellular analytic system (100) as described herein provides additional information on cell properties useful for differentiation of cells by mechanical strength. This information could be used for fine differentiation of similar or identical type of cells by genotype and defects due to genetic or metabolic deviations (such as maturity or end of life and etc.).

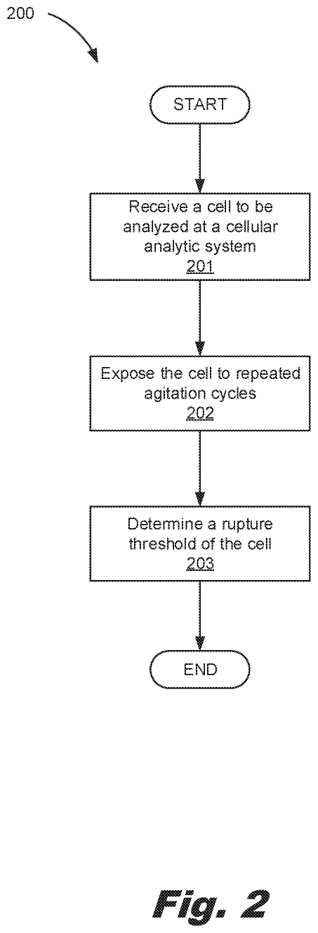

FIG. 2 is a flow chart of a method (200) of cell rupture threshold determination, according to an example of the principles described herein. As described above, in the method (200) a cell to be analyzed is received (block 201) at a cellular analytic system (FIG. 1, 100). That is, the cellular analytic system (FIG. 1, 100) may be fluidly coupled to a reservoir that holds a sample containing cells to be analyzed and these cells are introduced into the cellular analytic system (FIG. 1, 100). More specifically, the cells are received (block 201) in the chamber (FIG. 1, 104) of the analytic device (FIG. 1, 102).

In some examples, the cells may be received (block 201) serially. That is, each cell within the sample may be received (block 201) one at a time. Such a serial, single-file introduction of cells into the cellular analytic system (FIG. 1, 100) may be facilitated by having channels having a cross-sectional area size on the order of the cell diameter. Doing so facilitates the individual analysis of cells. Accordingly, rather than analyzing a portion of the sample and extrapolating therefrom, each cell of the sample may be analyzed. Thus, a complete analysis of the sample is performed.

Once in the cellular analytic system (FIG. 1, 100), the cell is exposed (block 202) to repeated agitation cycles. In some examples, the repeated agitation cycles may be performed by a single lysing element (FIG. 1, 106) or multiple lysing elements (FIG. 1, 106). In the case of a single lysing element (FIG. 1, 106), the cell may be looped past the single lysing element (FIG. 1, 106).

In the case of multiple lysing elements (FIG. 1, 106), the lysing elements (FIG. 1, 106) may be disposed (FIG. 1, 104) along a flow path through the chamber (FIG. 1, 104). Accordingly, as the cell passes along the flow path, it is exposed to the multiple distinct lysing elements (FIG. 1, 106).

In either case, the lysing intensity may increase or remain the same. For example, in a single lysing element (FIG. 1, 106) example, the lysing element (FIG. 1, 106) may be adjustable. As specific examples, the energy applied to a thermal inkjet resistor may increase or the intensity of ultrasonic waves may increase with each agitation cycle. In the example of multiple lysing elements (FIG. 1, 106), each distinct lysing element (FIG. 1, 106) may have a fixed intensity. For example, the energy applied to each thermal inkjet resistor may differ between different resistors, but the energy applied to a particular resistor may remain constant.

The cell is exposed (block 202) to the agitation cycles until the cell ruptures. As described above, cell rupture may be determined by the sensors (FIG. 1, 108) in the analytic device (FIG. 1, 102). That is, the sensor (FIG. 1, 108), be whatever type it may, can detect the difference between an intact cell and a ruptured cell.

The controller (FIG. 1, 110) of the cellular analytic system (FIG. 1, 100) then determines (block 203) based on a number of agitation cycles and a rupturing intensity of each agitation cycle, a rupture threshold of the cell. That is, the controller (FIG. 1, 110) relies on information passed from the sensors (FIG. 1, 108) to determine the rupture threshold. The information relied on may include, but is not limited to, a number of agitation cycles, an intensity of each agitation cycle, and whether or not a particular cell ruptured. That is, regardless of whether the agitation intensity increases or not, or whether the chamber (FIG. 1, 104) includes a single lysing element (FIG. 1, 106) or multiple lysing elements (FIG. 1, 106), the controller (FIG. 1, 110) can determine (block 203) the rupture threshold by knowing how many agitation cycles the cell was exposed to and the intensity of each agitation cycle. Thus, the controller (FIG. 1, 110) determines at what agitation intensity the cell ultimately ruptures. With such information on hand, the controller (FIG. 1, 110) can determine certain properties of the cell including cell type, cell state, etc.

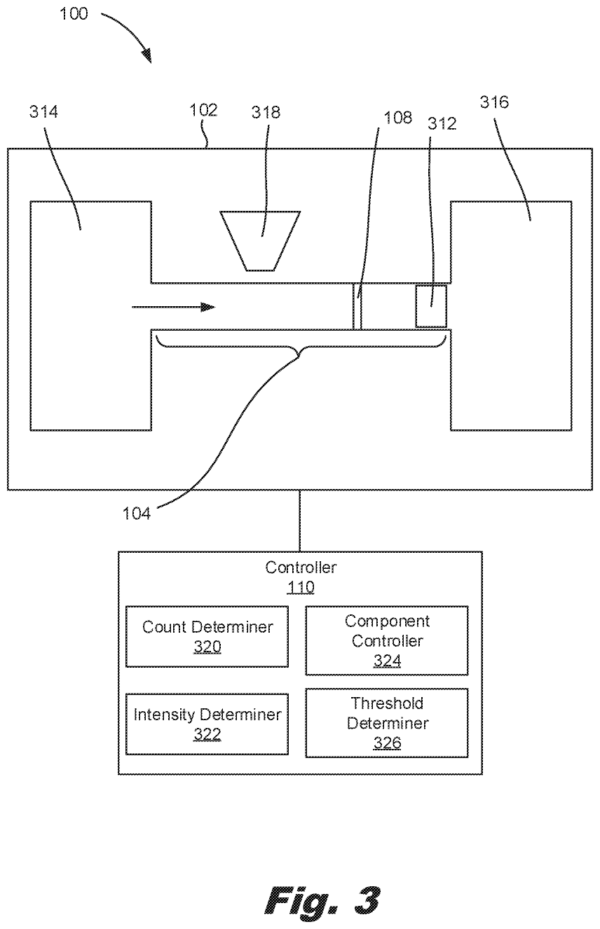

FIG. 3 is a diagrammatic representation of a cellular analytic system (100) for cell rupture threshold determination, according to an example of the principles described herein. Specifically, FIG. 3 depicts the analytic device (102) and the controller (110) that make up the cellular analytic system (100). IN the example depicted in FIG. 3, the analytic device (102) further includes a return pump (312) to return an un-ruptured cell to the chamber. In this example, the at least one lysing element (FIG. 1, 106) includes a single lysing element (FIG. 1, 106) that exposes the cell to repeated agitation cycles until the cell ruptures. In this example, the controller (110) determines the rupture threshold of the cell based on a count of how many agitation cycles the cell is exposed to until rupture.

FIG. 3 depicts an example where the chamber (104) includes a single lysing element (FIG. 1, 106). As described above, the cellular analytic system (100) receives a sample containing cells from an inlet (314). In some examples, the inlet (314) may be a reservoir or a channel from which the cells are received from another system. The cells to be analyzed may be of a variety of types such as cells cultured from plants, animals, or bacteria. The cells may be suspended in an appropriate extracellular fluid medium such as interstitial fluid and blood plasma.

In the example depicted in FIG. 3, the sample is moved along a flow path indicated by the arrow. The flow may be generated by an external pump. While along the fluid path, the cell is exposed to the activity of a lysing element (FIG. 1, 106) which in the example depicted in FIG. 3, is a lysing element (FIG. 1, 106) that is external to the chamber (104). However, in some examples, the lysing element (FIG. 1, 106) may be internal to the chamber (104).

As described above, the lysing element (FIG. 1, 106) may be of a variety of types. In the example depicted in FIG. 3, the lysing element (FIG. 1, 106) is an ultrasonic transducer (318) that generates high energy sonic waves. These high energy waves may travel through the wall of the chamber (104) to shear the cells disposed therein.

FIG. 3 also clearly depicts the sensor (108) that determines whether a cell has been sufficiently lysed. As a particular example, the sensor (108) may be an impedance sensor with metallic plates separated by a gap. A resistance between the plates can be determined. The measured resistance is indicative of a conductivity of the solution in the chamber (104). While FIG. 3 depicts a particular type of sensor (108), as noted above, other types of sensors may be implemented such as an optical scatter sensor, an optical fluorescence sensor, an optical bright field imaging system, an optical dark field imaging system, and a thermal property sensor.

As described above, the controller (110) may be used to determine whether a particular cell has been lysed. That is, the sensor (108) may provide a measurement, such as a conductivity measurement. The controller (110) can compare this value to a threshold value that maps to intact cells. Based on this comparison, it may be determined whether a cell membrane has ruptured or not.

Once lysed, the cells may be passed to an outlet (316). In some examples, the outlet (316) may be fluidly coupled to a downstream system for further analysis of the contents of the cell. In some examples, the outlet (316) may be a reservoir where the lysate fluid is contained.

As described above, the cellular analytic system (100) gradually increases the intensity of agitation such that it can be precisely determined at what stress level a particular cell ruptures. Increasing the agitation intensity may include increasing the intensity of the lysing element (FIG. 1, 106) and/or by increasing a count of how many exposures the cell has to the lysing element (FIG. 1, 106). For example, a lysing element (FIG. 1, 106) intensity may not change, but the cell may be passed by the lysing element (FIG. 1, 106) multiple times until cell rupture occurs. In another example, a lysing element (FIG. 1, 106) intensity increases with sequential agitation cycles and the cell may be passed by the lysing element (FIG. 1, 106) multiple times until cell rupture occurs.

Accordingly, in the example where the chamber (104) includes a single lysing element (FIG. 1, 106), the cellular analytic system (100) may include a return pump (312) to return an un-ruptured cell to be within the region of the single lysing element (FIG. 1, 106), i.e., the ultrasonic transducer (318) where the cell may be again exposed to the operation of the lysing element (FIG. 1, 106). This shifting of the un-ruptured cell to be by the lysing element (FIG. 1, 106) may be continued until the cell is ruptured, and thereby passed to the outlet (316).

Following each agitation cycle, certain information is passed to the controller (110). An intensity determiner (322) of the controller (110) determines an intensity of each lysing element (FIG. 1, 106) and whether that intensity changes or remains constant between agitation cycles. A count determiner (320) determines a count of the number of agitation cycles a cell is exposed to until it ruptures. Using this information, the threshold determiner (326) of the controller (110) can determine a strength of the cell, or a level of agitation that the cell can handle before rupture. That is, the threshold determiner (326) determines the rupture threshold of the cell based on a count of how many agitation cycles the cell is exposed to, and at what strength, before the cell ruptures. As described above, such information may be valuable in classifying and/or differentiating the cell from other cells of the same or differing types.

The controller (110) may include a processor and memory. The controller (110) may additionally include other electronics (not shown) for communicating with and controlling the various components of cellular analytic system (100), such as discrete electronic components and an ASIC (application specific integrated circuit). That is, in addition to determining a rupture threshold of the cell based on information received from the sensors (108), the controller (110) can control operation of the various components. For example, if the sensor (108) sends information to the controller (110) which indicates that a cell has not ruptured or is otherwise not sufficiently lysed, a component controller (324) of the controller (110) may activate the return pump (312) to push the cell against the flow path towards the lysing element (FIG. 1, 106) such that a second attempt at lysing can be carried out. By comparison, if the sensor (108) sends information to the controller (110) which indicates that a cell has ruptured, the component controller (324) may avoid activating the return pump (312) such that the cell can flow to the outlet (318).

As another example, the component controller (324) may increase the agitation intensity of the lysing element (FIG. 1, 106). For example, if the sensor (108) sends information to the controller (110) which indicates that a cell has not ruptured or is otherwise not sufficiently lysed, the component controller (324) may, in addition to activating the return pump (312) to push the cell in the path of the lysing element (FIG. 1, 106), increase the intensity of the lysing element (FIG. 1, 106) such that the agitation intensity for that agitation cycle is increased. Thus as described above, a cell is exposed to a gradual and incremental increase in agitation intensity such that an accurate and precise determination of the strength, or rupture threshold, of a particular cell can be determined, from which valuable information relating to the cell and its contents can be determined. Using a single lysing element (FIG. 1, 106) by which the cell passes multiple times may be beneficial in that it occupies a smaller footprint, thus allowing the placement of other lab-on-a-chip components.

For simplicity in the figures that follow certain specific components are omitted from illustration. However, these components, such as the components within the controller (110) may be implemented in any of the figures that follow.

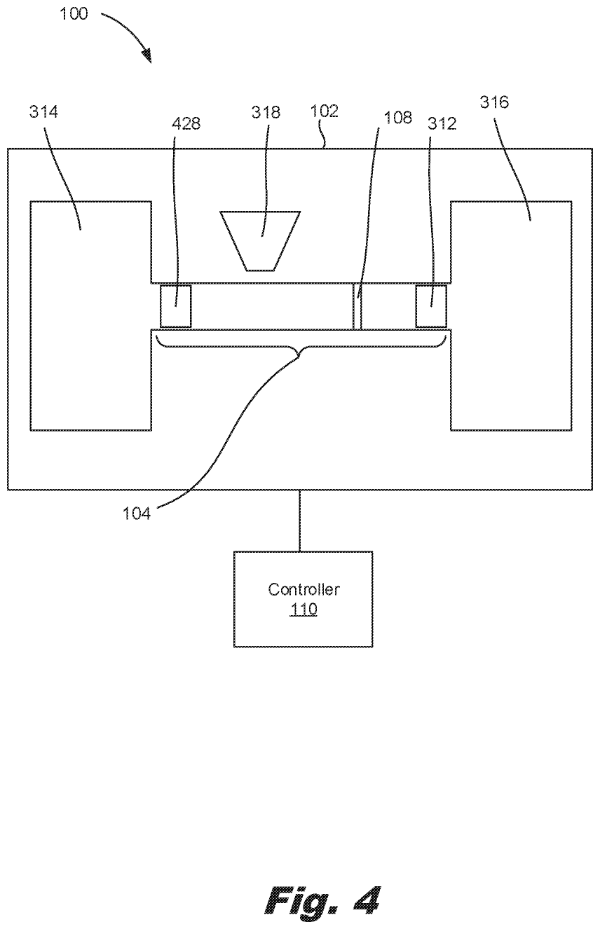

FIG. 4 is a diagrammatic representation of a cellular analytic system (100) for cell rupture threshold determination, according to another example of the principles described herein. Specifically, FIG. 4 depicts an example where the chamber (104) includes a single lysing element (FIG. 1, 106) which is depicted in FIG. 4 as an ultrasonic transducer (318). In the example depicted in FIG. 4, rather than relying on an external pump, the cellular analytic system (100) includes a main pump (428) to initiate flow through the chamber (104). In some examples, the main pump (428) may be an integrated pump, meaning the main pump (428) is integrated into a wall of the chamber (104). In some examples, the main pump (428) may be an inertial pump which refers to a pump which is in an asymmetric position within the inlet (314). The asymmetric positioning within the chamber (104) facilitates an asymmetric response of the fluid to the main pump (428). The asymmetric response results in fluid displacement when the main pump (428) is actuated. In some examples, the main pump (428) may be a thermal inkjet resistor, or a piezo-drive membrane or any other displacement device.

In this example, the controller (110) may control operation of the main pump (428). For example, if the sensor (108) sends information to the controller (110) which indicates that a cell has not ruptured or is otherwise not sufficiently lysed, the component controller (FIG. 3, 324) may de-activate the main pump (428) such that the return pump (312) may return the cell adjacent the lysing element (FIG. 1, 106). By comparison, if the sensor (108) sends information to the controller (110) which indicates that a cell has ruptured, the component controller (FIG. 3, 324) may leave the main pump (428) active, and maintain the return pump (312) inactive to drive the cell towards the outlet (316).

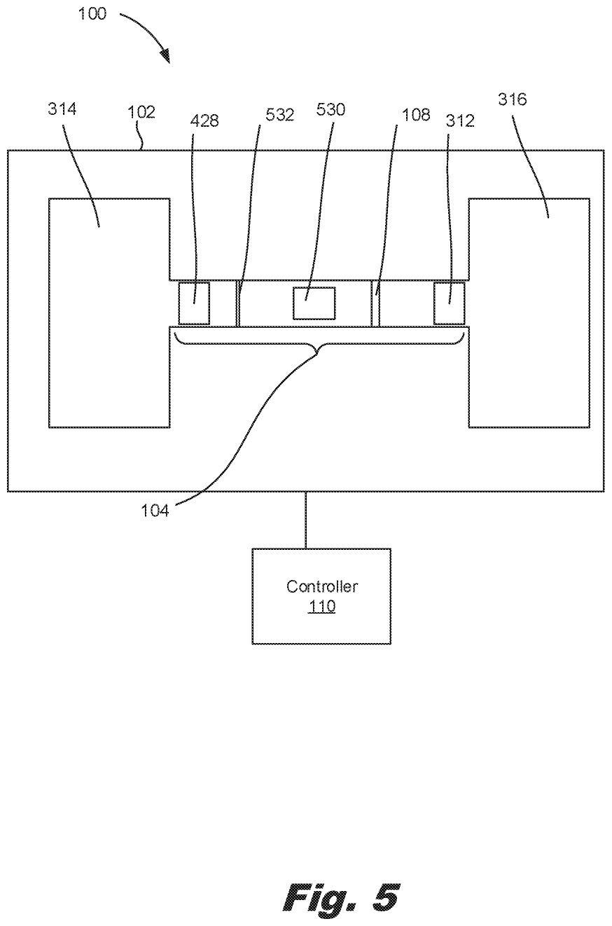

FIG. 5 is a diagrammatic representation of a cellular analytic system (100) for cell rupture threshold determination, according to another example of the principles described herein. FIG. 5 depicts some of the components described above such as the inlet (314), main pump (428), sensor (108), return pump (312), chamber (104), and outlet (316). FIG. 5 also depicts a lysing element (FIG. 1, 106). In this example, the lysing element (FIG. 1, 106) is a thermal inkjet resistor (530) that operates to generate a vapor bubble that shears a cell membrane as described above.

FIG. 5 also depicts a cell presence sensor (532). The cell presence sensor (532) may be of any variety of types such as those described as examples of the sensor (108). That is, the cell presence sensor (532) may be an impedance sensor, an optical scatter sensor, an optical fluorescence sensor, an optical bright field imaging system, an optical dark field imaging system, or a thermal property sensor. This cell presence sensor (532) is disposed before the lysing element (FIG. 1, 106) and may trigger activation of the lysing elements (FIG. 1, 106). For example, if the cell presence sensor (532) sends information to the controller (110) which indicates that a cell is not present within the chamber (104), the component controller (FIG. 3, 324) may avoid activating the lysing element (FIG. 1, 106). By comparison, if the cell presence sensor (532) sends information to the controller (110) which indicates that a cell is present, the component controller (FIG. 3, 324) may activate the lysing element (FIG. 1, 106). FIG. 5 depicts a cell presence sensor (532) per lysing element (FIG. 1, 106), other configurations are possible as well. That is, the analytic device (102) includes a single cell presence sensor (532) may detect the presence of a cell within the chamber and all lysing elements (FIG. 1, 106) are activated based on a determined cell presence by the single cell presence sensor (532). In another example, the analytic device (102) includes a cell presence sensor (532) per lysing element (FIG. 1, 106) to detect the presence adjacent a respective lysing element (FIG. 1, 106) and the lysing element (FIG. 1, 106) is activated based on a determined cell presence by the respective cell presence sensor (532).

adjacent a single lysing element (FIG. 1, 106) or multiple cell presence sensors (532) may detect the presence of a cell adjacent a respective lysing elements (FIG. 1, 106) and the component controller (FIG. 3, 324) may activate/deactivate each lysing elements (FIG. 1, 106) based on an output of a corresponding cell presence sensor (532).

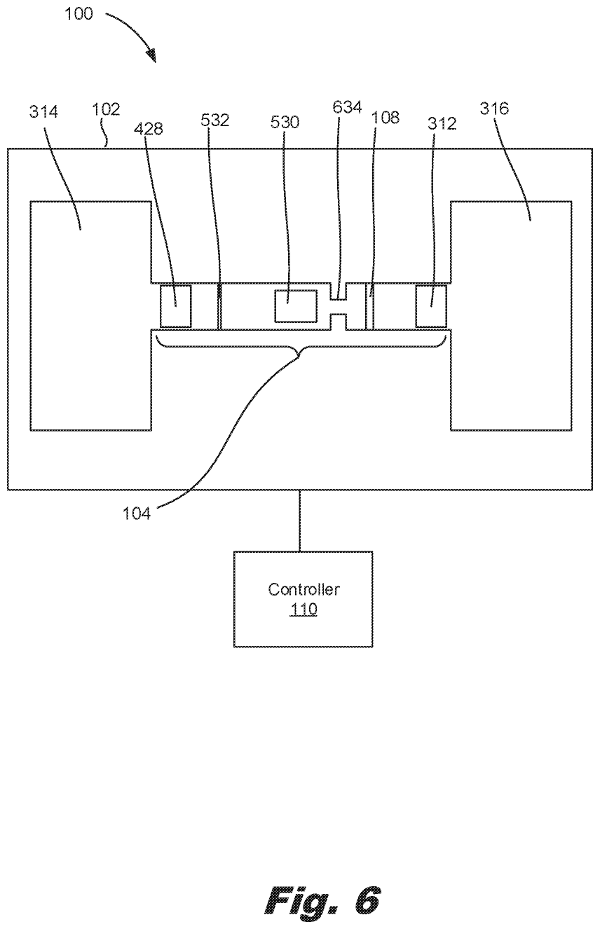

FIG. 6 is a diagrammatic representation of a cellular analytic system (100) for cell rupture threshold determination, according to another example of the principles described herein. FIG. 6 depicts some of the components described above such as the inlet (314), main pump (428), the cell presence sensor (532), the thermal inkjet resistor (530), sensor (108), return pump (312), chamber (104), and outlet (316). In this example, the chamber (104) includes a second lysing element (FIG. 1, 106) in the form of a constriction (634) within the chamber (104). The constriction (634) increases pressure within the fluid, which can cause the cell membrane/wall to rupture.

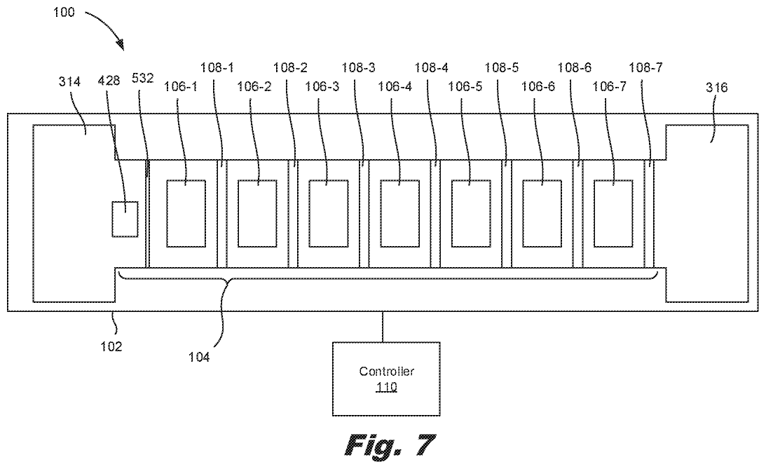

FIG. 7 is a diagrammatic representation of a cellular analytic system (100) for cell rupture threshold determination, according to another example of the principles described herein. In the example depicted in FIG. 7, the chamber (104) includes multiple sequential lysing elements (106) and at least one sensor (108) per lysing element (106). Associated with each lysing element (106) is a sensor (108). Each sensor (108) detects whether a cell rupture occurred. That is, in the example depicted in FIG. 7, rather than passing a cell by a single lysing element (106) multiple times, the cell is passed by multiple lysing elements (106) a single time. This example may be beneficial in that it alleviates certain controlled elements such as the return pump (FIG. 3, 312). While FIG. 7 depicts seven lysing elements (106-1, 106-2, 106-3, 106-4, 106-5, 106-6, 106-7) and seven sensors (108-1, 108-2, 108-3, 108-4, 108-5, 108-6, 108-7) any number of lysing elements (106) and sensors (108) may be disposed within the cellular analytic system (100).

In this example, information from each of the sensors (108) is passed to the controller (110) for cell rupture threshold determination. That is, as a cell passes by each of the sensors (108), information is passed to the controller (110) to determine whether a corresponding lysing element (106) ruptured the cell. With this information, the controller (110) can determine a rupture threshold, or strength of a cell. That is, based on sensor (108) outputs, the controller (110) can determine how far down the flow path the cell gets before rupture occurs. For example, if a fifth sensor (108-5) passes information consistent with an intact cell, but the sixth sensor (108-6) passes information consistent with a ruptured cell, the controller (110) determines that six agitation cycles at a certain intensity resulted in cell rupture. In the example depicted in FIG. 7, the lysing elements (106) may have the same agitation intensity, however in other examples as depicted further below, the lysing elements (106) may have different intensities.

In addition to determining a cell rupture threshold, the controller (110) also controls various components of the cellular analytic system (100). For example, the component controller (FIG. 3, 324) may independently activate/deactivate certain of the lysing elements (106). For example, a particular lysing element (106) may be activated/deactivated based on lysing results of an earlier lysing element. For example, the component controller (FIG. 3, 324) may activate a downstream lysing element (106) based on the lysing results of an upstream lysing element (106). As a specific example, a second sensor (108-2) may determine that a cell is un-ruptured by the second lysing element (106-2). Accordingly, the component controller (FIG. 3, 324) may activate the third lysing element (106-3). In another example, the component controller (FIG. 3, 324) may deactivate a downstream lysing element (106) based on the lysing results of an upstream lysing element (106). As a specific example, a third sensor (108-3) may determine that a cell is ruptured by the third lysing element (106-3). Accordingly, the component controller (FIG. 3, 324) may deactivate the fourth and subsequent lysing elements (106-4, 106-5, 106-6).

FIG. 7 also depicts the example where a single cell presence sensor (532) triggers activation of each of the downstream components. That is, in this example, once the cell presence sensor (532) detects the presence of a cell, downstream lysing elements (106) may be activated to agitate the cell.

Thus, the example depicted in FIG. 7 with multiple lysing elements (106) in the chamber (104) facilitate the sequential and gradual increase in intensity of a lysing energy such that a determination of a rupture threshold for different cells can be made at a high resolution.

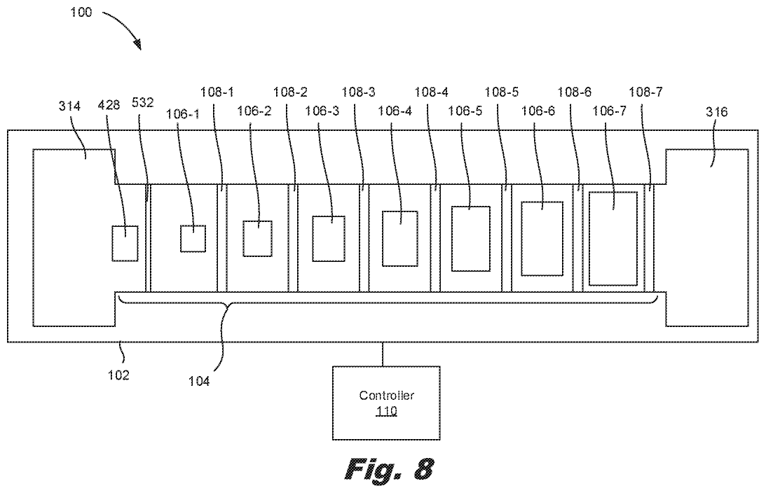

FIG. 8 is a diagrammatic representation of a cellular analytic system (100) for cell rupture threshold determination, according to another example of the principles described herein. FIG. 8 is an example where the chamber (104) includes multiple lysing elements (106) each with a corresponding sensor (108). However, in the example depicted in FIG. 8, the lysing elements (106) are different from one another. The lysing elements (106) may be different in any number of ways. For example, the lysing elements (106) may exhibit increasing agitation intensities. In some examples, the progression of agitation intensity coincides with the flow path of a cell through the chamber (104). That is, as the cell moves along the flow path it is sequentially exposed to increasingly intense lysing energies.

The different lysing intensities may be manifest in different ways. In one example, each lysing element (106) may be of a similar type, but of different strengths. For example, each lysing element (106) may be a thermal inkjet resistor (FIG. 5, 530), but of different sizes as depicted in FIG. 8. In this example, larger thermal inkjet resistors (FIG. 5, 530) generate larger vapor bubbles thus generating a stronger shear force against the cell. In another example, a larger voltage may be placed across thermal inkjet resistors (FIG. 5, 530) of the same size. This increases the bubble generation and thus the shear rate on the cell.

In another example, where the lysing elements (106) are ultrasonic transducers (FIG. 3, 318), the different transducers (FIG. 3, 318) may emit waves of different frequency and/or amplitude in order to increase the agitation intensity. In yet another example, the lysing elements (106) may be of different types. For example some chambers may include thermal inkjet resistors (FIG. 5, 530), and others may include ultrasonic transducers (FIG. 5, 530) while yet others are non-reversible electroporation electrodes.

As with the example depicted in FIG. 7, information from each of the sensors (108) is passed to the controller (110) for cell rupture threshold determination. That is, as a cell passes by each of the sensors (108), information is passed to the controller (110) to determine whether a corresponding lysing element (106) ruptured the cell. With this information, the controller (110) can determine a rupture threshold, or strength of a cell. That is, based on sensor (108) outputs, the controller (110) can determine how far down the flow path the cell gets before rupture occurs.

Thus, the example depicted in FIG. 8 with multiple lysing elements (106) in the chamber (104) facilitate the sequential and gradual increase in intensity of a lysing energy such that a determination of a rupture threshold for different cells can be made at a high resolution.

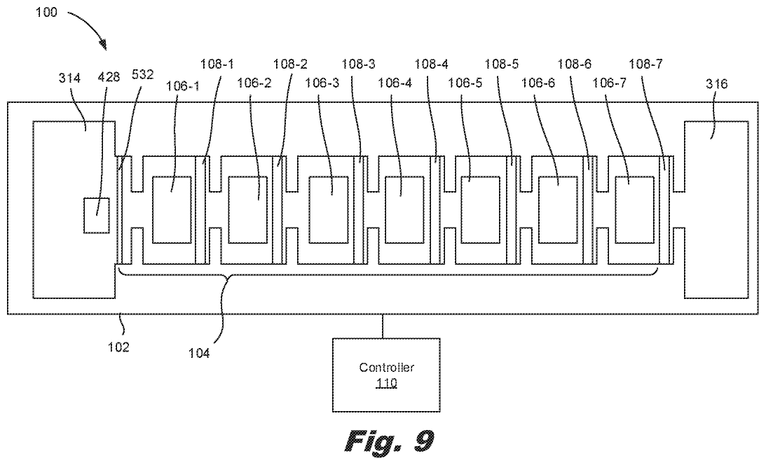

FIG. 9 is a diagrammatic representation of a cellular analytic system (100) for cell rupture threshold determination, according to another example of the principles described herein. As with the above figures, FIG. 9 depicts a cellular analytic system (100) with an inlet (314), a chamber (104) with multiple lysing elements (106) and sensors (108), a cell presence sensor (532) and an outlet (316). In the example depicted in FIG. 9, the cellular analytic system (100) also includes a main pump (428) to move fluid through the chamber (104). In the example depicted in FIG. 9, the chamber (104) includes a constriction (FIG. 6, 634) between adjacent lysing elements (106). In FIG. 9, reference numbers for the constrictions (FIG. 6, 634) are omitted for visual clarity. In one example, sequential lysing elements (106) exhibit increasing agitation intensities.

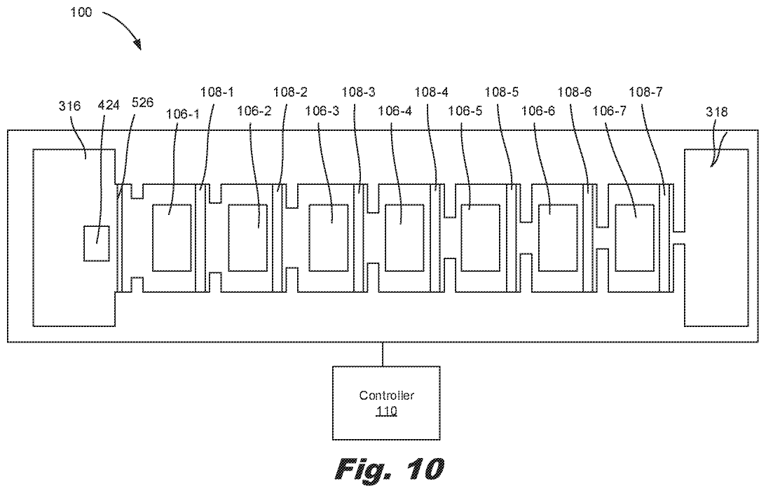

As stated above, the constrictions (FIG. 6, 634) provide another lysing structure to potentially rupture the cell membrane. As depicted in FIG. 10, in some examples, the constrictions (FIG. 6, 634) change in size. For example, as depicted in FIG. 10, the constrictions (FIG. 6, 634) get narrower along the flow path. The changing size of the constrictions (FIG. 6, 634) is yet another way in which the agitation intensity rises along the flow path. That is, the narrower the constriction (FIG. 6, 634) the more stress placed upon the cell passing there through. While FIGS. 9 and 10 depict constrictions (FIG. 6, 634) with lysing elements (106) of the same size and/or type, such constrictions (FIG. 6, 634) may be implemented in systems where a type and/or intensity of the lysing element (106) changes along the flow path. Thus, the present cellular analytic system (100) provides a system wherein cells can be exposed to incremental agitation stresses to determine their precise rupture threshold. The resolution of the cellular analytic system (100) may be determined by differences in sizes of the thermal inkjet resistors (FIG. 5, 530), difference in constriction (FIG. 6, 634) size, and/or difference in levels of ultrasound energy intensity.

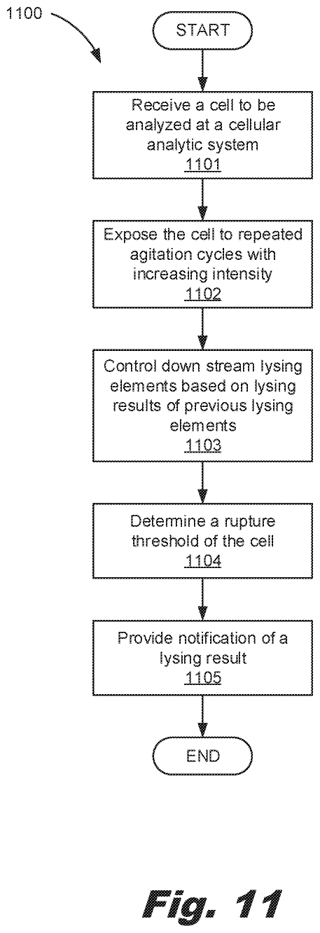

FIG. 11 is a flow chart of a method (1100) off cell rupture threshold determination, according to another example of the principles described herein. A cell to be analyzed is received (block 1101) at a cellular analytic system (FIG. 1, 100) and exposed (block 1102) to repeated agitation cycles with increasing intensity. In some examples this may be performed as described above in connection with FIG. 2.

In some examples, downstream lysing elements (FIG. 1, 106) are controlled (block 1103) based on lysing results of previous lysing elements (FIG. 1, 106). For example, if it is determined that a particular cell has not ruptured, downstream lysing elements (FIG. 1, 106) may be activated or remain in their existing active state. In another example, if it is determined that a particular cell has ruptured, downstream lysing elements (FIG. 1, 106) may be deactivated. Such deactivation of downstream lysing elements (FIG. 1,106) conserve energy as lysing elements are not needlessly operated as a cell is already lysed. Moreover, deactivating the lysing elements (FIG. 1, 106) may preserve the sample. That is, additional lysing operations may contaminate, alter, or otherwise harm a lysed sample.

With information collected from the various sensors (FIG. 1, 108) of the cellular analytic system (FIG. 1, 100), a rupture threshold for the cell is determined (block 1104). This may be performed as described above in connection with FIG. 2. In some examples, the method (1100) includes providing (block 1105) a notification of a lysing result for each lysing element (FIG. 1, 106). That is, if a particular lysing element (FIG. 1, 106) successfully lyses the cell, a notification may be provided (block 1105). The notification may be of varying types and complexity. For example, the notification may be a printout indicating the cells rupture threshold or strength. In another example, the notification may include detailed information on each agitation cycle. Such notification provides even more information to an operator regarding the viability or strength of a cell such that subsequent cellular analysis can be carried out.

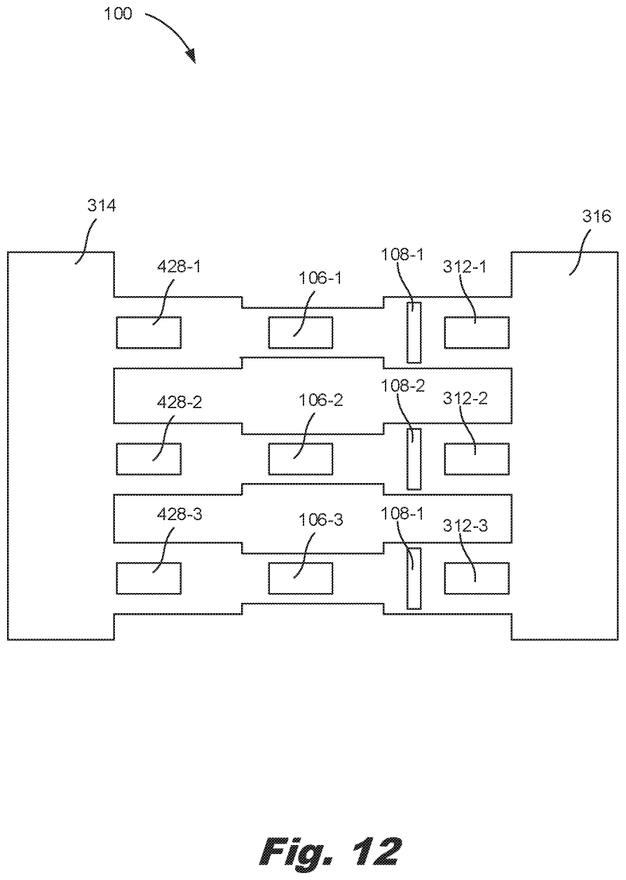

FIG. 12 is a diagrammatic representation of a cellular analytic system (100) for cell rupture threshold determination, according to another example of the principles described herein. In the example depicted in FIG. 12, the cellular analytic system (100) includes multiple parallel chambers (FIG. 1, 104). The multiple parallel chambers (FIG. 1, 104) facilitate the processing of more cells. For example, rather than analyzing a single cell at a time, the cellular analytic system of FIG. 12 includes three chambers (FIG. 1, 104) and three respective main pumps (428-1, 428-2, 428-3), lysing elements (106-1, 106-2, 106-3), sensors (108-1, 108-2, 108-3), and return pumps (312-1, 312-2, 312-3) to process three cells at the same time as they pass from the inlet (314) to the outlet (316). Note that in this example, the different distinct lysing elements (106) may be of different types and/or sizes. While FIG. 12 depicts an example with one lysing element (106) per chamber (FIG. 1, 104), in some examples, the parallel chambers may have multiple lysing elements (106) as depicted in FIGS. 7-10.

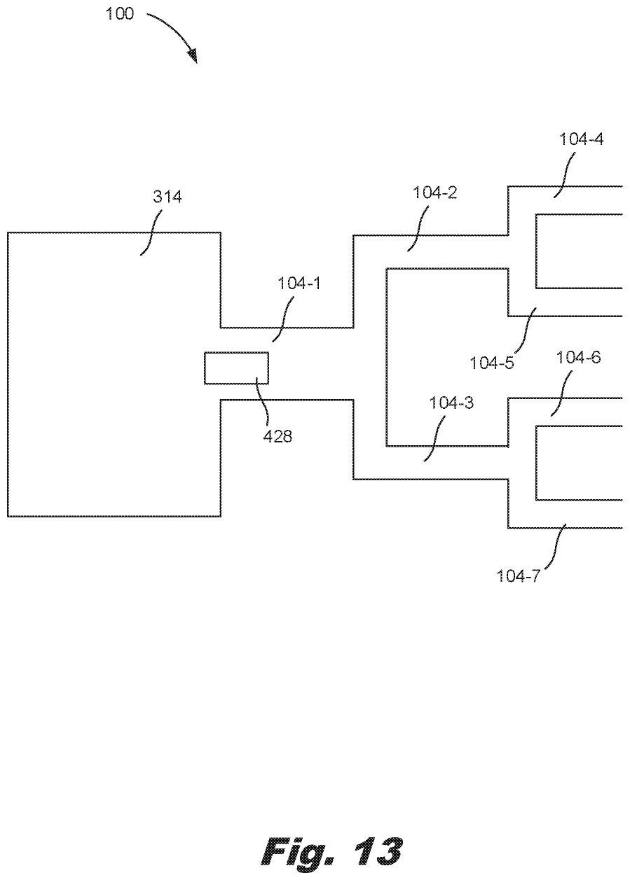

FIG. 13 is a diagrammatic representation of a cellular analytic system (100) for cell rupture threshold determination, according to another example of the principles described herein. In this example, the cellular analytic system (100) includes branched chambers (104-1, 104-2, 104-3, 104-4, 104-5, 104-6, 104-7). Note that as the fluid passes to each chamber (104) a cross-sectional area of the chamber (104) decreases. In this example, the reduction in the cross-sectional are of the chamber (104) acts as the agitation mechanism with increasingly smaller cross-sectional areas representing an increased lysing intensity. Using branched chambers (104) as depicted in FIG. 13 may reduce the overall fluidic resistance of the cellular analytic system (100) such that a lower pressure main pump (428) may be implemented.

In summary, using such a cellular analytic system 1) allows analysis of the cell rupture threshold; 2) leads to cell classification/differentiation based on the cell rupture threshold; 3) automates precision separation and cytometry 4) can be integrated onto a lab-on-a-chip device; 5) is scalable and can be parallelized, and 6) is low cost and effective. However, the devices disclosed herein may address other matters and deficiencies in a number of technical areas.

What is claimed is:

1. A cellular analytic system, comprising:
an analytic device, comprising:
a chamber to receive a cell to be analyzed;
at least one lysing element to agitate the cell; and
at least one sensor to detect a change in the cell based on an agitation of the cell; and
a controller to determine a rupture threshold of the cell based on parameters of the agitation when a cell membrane ruptures.

2. The system of claim 1, wherein:
the analytic device further comprises a return pump to return an un-ruptured cell to the chamber;
the at least one lysing element comprises a single lysing element to expose the cell to repeated agitation cycles until the cell ruptures; and
the controller determines the rupture threshold of the cell based on a count of how many agitation cycles the cell is exposed to until rupture.

3. The system of claim 2, wherein the single lysing element is to increase an agitation intensity with sequential agitation cycles.

4. The system of claim 1, wherein the chamber comprises:
multiple sequential lysing elements; and
at least one sensor per lysing element.

5. The system of claim 4, further comprising at least one of:
a constriction between adjacent lysing elements; and
sequential lysing elements exhibit increasing agitation intensities.

6. The system of claim 1, wherein:
the at least one sensor is selected from the group consisting of:
an impedance sensor;
an optical scatter sensor;
an optical fluorescence sensor;
an optical bright field imaging system;
an optical dark field imaging system; and
a thermal property sensor;
the at least one lysing element is selected from the group consisting of:
a thermal inkjet heating resistor;
a non-reversible electroporation electrode;
a piezo-electric device; and
an ultrasonic transducer.

7. A method, comprising:
receiving a cell to be analyzed at a cellular analytic system;
exposing the cell to repeated agitation cycles with increasing intensity until the cell ruptures; and
determining, based on a number of agitation cycles and an intensity of each agitation cycle, a rupture threshold of the cell.

8. The method of claim 7, further comprising controlling downstream lysing elements of the cellular analytic system based on a lysing result of a previous lysing element.

9. The method of claim 7, wherein exposing the cell to repeated agitation cycles comprises looping the cell past a single lysing element.

10. The method of claim 7, wherein exposing the cell to repeated agitation cycles comprises moving the cell past multiple lysing elements.

11. The method of claim 7, further comprising providing a notification of a lysing result of each lysing element.

12. A cellular analytic system, comprising:
an analytic device, comprising:
at least one chamber to receive a cell to be analyzed;
at least one lysing element; and
at least one sensor per lysing element to determine when the cell ruptures, wherein the analytic device repeatedly agitates the cell with increasing intensity until the cell ruptures;
a main pump to move the cell through the analytic device; and
a controller comprising:
a count determiner to determine a number of agitation cycles until cell rupture;
an intensity determiner to determine an intensity of each of the number of agitation cycles;
a threshold determiner to determine a rupture threshold of the cell based on the number of agitation cycles until cell rupture and the intensity of each of the number of agitation cycles; and
a component controller to alter operation of at least one component of the analytic device based on a cell rupture.

13. The system of claim 12, wherein the at least one chamber comprises at least one of:
multiple parallel chambers; and
branched chambers.

14. The system of claim 12, wherein:
the analytic device comprises a single cell presence sensor to detect a cell presence within the chamber; and
all lysing elements are activated based on a determined cell presence by the single cell presence sensor.

15. The system of claim 12, wherein:
the analytic device comprises a cell presence sensor per lysing element to detect a cell presence adjacent a respective lysing element; and
a lysing element is activated based on a determined cell presence by a respective cell presence sensor.
